(19) United States
(12) Patent Application Publication
(10) Pub. No.: US 2007/0134156 A1
(43) Pub. Date: Jun. 14, 2007

(54) Fatigue evaluation apparatus, fatigue evaluation method, and application thereof

(75) Inventors: Kei Mizuno, Osaka-shi (JP); Satoshi Nozaki, Osaka-shi (JP); Koji Yamaguchi, Kyoto-shi (JP); Hiroshi Mizuma, Sakai-shi (JP); Masaaki Tanaka, Osaka-shi (JP); Tetsuya Sasabe, Kawachinagano-shi (JP); Hirohiko Kuratsune, Toyonaka-shi (JP); Yasuyoshi Watanabe, Minoo-shi (JP); Suzuka Ataka, Osaka-shi (JP); Yasushi Nishida, Kashiwa-shi (JP)

Correspondence Address: SHERIDAN ROSS PC, 1560 BROADWAY, SUITE 1200, DENVER, CO, 80202, US

(73) Assignees: SOIKEN INC., OSAKA (JP); YASUYOSHI WATANABE, OSAKA (JP); HIROKIHO KURATSUNE, OSAKA (JP)

(21) Appl. No.: 10/581,565

(22) PCT Filed: Feb. 7, 2005
(86) PCT No.: PCT/JP05/01790

§ 371 Date: Nov. 14, 2006

(30) Foreign Application Priority Data
Feb. 17, 2004 (JP) 75324/2004

(51) Int. Cl. A61K 49/00; A61K 31/198; G01N 33/00

(52) U.S. Cl. 424/9.2; 514/561; 436/96; 436/106

(57) ABSTRACT

The present invention provides a method for easily and quantitatively evaluating a degree of fatigue of a human being, a kit therefor, and an application thereof. Specifically, by measuring a concentration of amino acid in plasma taken from a subject, the degree of fatigue of the subject in daily life can be easily and quantitatively evaluated. Furthermore, an in vivo anti-fatigue effect of an anti-fatigue substance and that of an anti-fatigue food product can be measured.

### TECHNICAL FIELD

The present invention relates to a method for evaluating the degree of fatigue in a human being and an application thereof. More particularly, the present invention relates to a method for evaluating the degree of fatigue of a human being by using as an index a change in concentration of at least one of amino acids in a body fluid, for example, total amino acids, branched-chain amino acids, aromatic amino acids, cysteine, methionine, lysine, arginine, and histidine, and an application thereof.

### BACKGROUND ART

Fatigue is a very immediate problem in daily life, and many people living in a stressful modern world suffer from chronic fatigue. However, scientific and medical research into “fatigue” has been carried out only fragmentally, and almost no research has been carried out into determinant means or a quantitative measure which shows how to objectively and quantitatively express a subjective symptom called “fatigue”.

Up to now, research has been carried out mainly into muscle fatigue as a typical example of “fatigue”, and attention has been paid to, as an index of muscle fatigue, an increase in amount of lactate production. However, lactic acid is basically an important energy source for a central nervous system, and a theory that lactic acid inhibits muscular activity is now viewed in a negative light. Further, it has become clear that branched-chain amino acids serve for an exercise endurance maintenance mechanism under exercise stress and are consumed in a muscle and, as a result, that branched-chain amino acids in plasma decreases. However, a mechanism of mental fatigue has not been made clear (see Non-patent Document 1). Furthermore, a phenomenon that muscle fatigue causes pyruvic acid in a body fluid to increase and causes a pH value in the body fluid to drop is known. This phenomenon can be observed when a certain amount of stress is given as exercise stress to the muscle, but “fatigue” is different from local muscle fatigue and is considered to be a broader physiological phenomenon which can be observed in a living organism.

Further, over the few years, more than 8,000 people have been killed and more than 100,000 people have been injured annually in traffic accidents (see Non-patent Document 2), and fatigue due to driving of motor vehicles is considered to be a factor of these traffic accidents. Further, a driver of an automobile or other types of vehicle is exemplified as an occupation which poses a high cardiovascular risk and a high risk of death (see Non-patent Document 3), and overwork is considered to be an important risk factor of death. Thus, a problem of fatigue due to driving is very important medically, socially, and economically. Nevertheless, almost no research has been carried out into driving and fatigue and, in particular, into prevention and reduction of fatigue due to driving of an automobile. Furthermore, there are only undeveloped measures against the problem of fatigue due to driving.

[Patent Document 1]

Japanese Unexamined Patent Publication No. 026987/1996 (Tokukaihei 08-026987; published on Jan. 30, 1996).

[Non-patent Document 1]

H. K. Struder, W. Hollman, P. Platen, R.Wostmann, H. Weicker, and G. J. Molderings: “Effect of acute exercise on plasma amino acids and prolactin concentrations and on [³H]ketanserin binding to serotonin2A receptors on human platelets”, Eur J Appl Physiol, 1999.

[Non-patent Document 2)

Traffic Planning Division of the Traffic Bureau of the National Police Agency: Annual Report on Traffic Accident Statistics.

[Non-patent Document 3]

T. Uehata: A Study of Overwork, p. 1-190, 1993.

As described above, methods for objectively determining fatigue due to exercise stress have been proposed. However, although many Japanese people feel fatigue symptoms in daily life as described above, only a few methods for objectively evaluating the fatigue symptoms have been reported. Further, the fatigue symptoms in daily life, if neglected, may lead directly to overwork death, which means sudden death caused by overworking for many hours. Furthermore, although a problem of overwork death is recognized as a very important medical, economical, and social issue, little is known about a scientific mechanism of overwork death. In order to prevent overwork death, which has recently been recognized as a social problem, there is a demand for a method for objectively evaluating the degree of fatigue.

Further, because each of many pharmaceutical products and health food products such as nutrition-supplement drinks that have become prevalent in the market features a function of treating or inhibiting fatigue, a scientific basis of the function has been widely demanded by consumers, the market, and society as a whole.

As described above, although there is some knowledge of fatigue due to exercise stress, fatigue due to exercise stress is totally different from mental fatigue in daily life, and a method for evaluating mental fatigue in daily life has not been developed. For this reason, there has been a strong demand for development of a method for easily and objectively evaluating in vivo mental fatigue in daily life and an application thereof. The present invention has been made in view of the foregoing problems and has as an object to provide a method for easily and quantitatively evaluating the degree of fatigue or, in particular, mental fatigue and an application thereof.

### DISCLOSURE OF INVENTION

As a result of diligently studying in consideration of the foregoing problems, the inventors have uniquely found that the degree of fatigue in daily life or, in particular, the degree of fatigue with respect to mental fatigue loading can be evaluated quantitatively by simply measuring and evaluating a change in concentration of amino acid in a body fluid. The inventors have completed the present invention which makes it possible to measure a degree of mental fatigue in daily life by using such an experimental system.

A fatigue evaluation apparatus according to the present invention is arranged so as to include: measuring means for measuring a concentration of amino acid in a body fluid; and evaluating means for evaluating a degree of fatigue by using as an index a measurement result obtained by the measuring means.

Further, it is preferable that when the measurement result shows that the concentration of the amino acid is lower than a predetermined value, the evaluating means determine that the degree of fatigue is high.

Further, it is preferable that when the measurement result shows that the concentration of the amino acid is lower than the predetermined value, the evaluating means determine that there is an overwork state due to accumulation of physiological acute fatigue developed in daily life.

Further, it is preferable that the body fluid be at least one type of body fluid selected from a group consisting of plasma, saliva, cerebrospinal fluid, and urine, all of which have been separated from an individual organism.

Further, it is preferable that the amino acid be at least on type of amino acid selected from a group consisting of total amino acids, branched-chain amino acids, aromatic amino acids, cysteine, methionine, lysine, arginine, and histidine.

Further, it is preferable that a target for evaluation of the degree of fatigue be physiological acute fatigue developed in daily life, particularly mental fatigue.

Further, it is preferable that the measuring means measure respective concentrations of the amino acid in the body fluid before and after a subject is subjected to fatigue loading, and the evaluating means evaluate the degree of fatigue by using as an index a change in concentration of the amino acid in the body fluid between before and after the fatigue loading, based on the measurement result obtained by the measuring means.

Further, in order to solve the foregoing problems, a fatigue evaluation method according to the present invention is arranged so as to evaluate a degree of fatigue by using as an index a concentration of amino acid in a body fluid. According to the foregoing method, a degree of fatigue of a human being can be easily and quantitatively evaluated. The method also makes it possible to quantitatively measure an effect of a pharmaceutical product having a fatigue-treating or -inhibiting effect and that of a nutraceutical product such as a nutrition-supplement drink or a health food product. Furthermore, the method also makes it possible to easily and objectively detect an overwork state easily caused by excessive working hours.

Further, it is preferable that when the concentration of the amino acid is low, it be determined that the degree of fatigue is high. Further, it is preferable that when the concentration of the amino acid is low, it be determined that there is an overwork state due to accumulation of physiological acute fatigue developed in daily life. Further, it is preferable that the body fluid be at least one type of body fluid selected from plasma, saliva, cerebrospinal fluid, and urine. Further, it is preferable that the amino acid be at least one type of amino acid selected from total amino acids, branched-chain amino acids, aromatic amino acids, cysteine, methionine, lysine, arginine, and histidine. Further, it is preferable that a target for the degree of fatigue be physiological acute fatigue developed in daily life, particularly mental fatigue. Further, it is preferable that the degree of fatigue be evaluated by using as an index a change in concentration of the amino acid in the body fluid between before and after fatigue loading.

Further, in order to solve the foregoing problems, a fatigue evaluation kit according to the present invention is arranged so as to carry out the foregoing fatigue evaluation method.

According to the foregoing fatigue evaluation kit, for example, by simply measuring and calculating a concentration of amino acid in a body fluid taken from a subject, an effect of a pharmaceutical product having a fatigue-inhibiting or -treating effect and that of a food product having the same effect can be evaluated. That is, an in vivo effect of a pharmaceutical product or a food product having a fatigue-inhibiting or -treating effect can be easily and quantitatively measured.

Further, in order to solve the foregoing problem, a method according to the present invention for measuring an anti-fatigue effect of an anti-fatigue substance is arranged so as to measure the anti-fatigue effect of the anti-fatigue substance by using any one of the foregoing fatigue evaluation method and the foregoing fatigue evaluation kit.

According to the foregoing method, it is possible to easily, securely, and quantitatively measure a degree to which an anti-fatigue substance treats a fatigue symptom of a human being, i.e., an anti-fatigue effect of an anti-fatigue substance.

The present invention provides a method for easily and quantitatively measuring and evaluating a degree of fatigue in daily life, a kit therefor, and an application thereof. Thus, the present invention makes it possible to objectively find a degree of fatigue in daily life so as to avoid various diseases caused by unconscious accumulation of fatigue. Furthermore, the present invention makes it possible to lower an incidence of overwork death caused by continuing to work without noticing fatigue.

Further, a method according to the present invention is a method for evaluating an anti-fatigue effect of an anti-fatigue substance, the method including the processes of: administering the anti-fatigue substance to a subject in a fatigue state; determining whether or not the subject has recovered from fatigue, by using any one of the foregoing fatigue evaluation apparatus, the foregoing fatigue evaluation method, and the foregoing fatigue evaluation kit; and evaluating the anti-fatigue effect of the anti-fatigue substance by using as an index a degree to which the subject has recovered from fatigue.

Further, an anti-fatigue effect evaluation system according to the present invention is arranged so as to include: a first fatigue evaluation apparatus according to the foregoing fatigue evaluation apparatus for evaluating a degree of fatigue of a subject to whom an anti-fatigue substance has been administered; and a second fatigue evaluation apparatus for evaluating an anti-fatigue effect of the anti-fatigue substance by using as an index a degree to which the subject has recovered from fatigue, based on an evaluation result of the first fatigue evaluation apparatus.

Further, a screening method according to the present invention is a method for screening a candidate substance for an anti-fatigue substance, the method including the processes of: administering a test article to a model animal in a fatigue state; determining whether or not the model animal has recovered from fatigue, by using any one of the foregoing fatigue evaluation apparatus, the foregoing fatigue evaluation method, and the foregoing fatigue evaluation kit; and determining that the test article is a candidate substance for an anti-fatigue substance, by using as an index the model animal's recovery from fatigue.

The foregoing fatigue evaluation apparatus may be achieved by a computer. In this case, a fatigue evaluation apparatus control program which achieves the forgoing fatigue evaluation apparatus by causing the computer to operate each of the foregoing means, and a computer-readable storage medium which stores the control program are also included in the scope of the present invention.

Furthermore, the present invention makes it possible to provide to consumers and society information on to what extent a variety of pharmaceutical products and food products supplied to the market and featuring a fatigue-treating effect, nutritional fortification, and nutritional support exert in vivo anti-fatigue effects. These pieces of information help the consumers to choose an anti-fatigue food product and an anti-fatigue pharmaceutical product effective in prevention of overwork and in nutritional fortification. In these respects, the present invention is very useful and has a strong social impact.

### BRIEF DESCRIPTION OF DRAWINGS

FIG. 1 is an explanatory diagram of “mirror drawing test,” which is one of mental fatigue loading methods in Example 1 according to the present invention. The drawing of mirrored characters is performed in such a manner that a mirror reversed state (22) of test characters which are written on a sheet (21) of paper and which are reflected on a mirror (23) is directly copied onto another piece of paper.

FIG. 2 shows a VAS test paper used in [1-4]. The figure shown herein reflects a full scale of a line segment in the VAS test paper, and the line segment is normally in the order of 10 cm long.

FIG. 3 is a graph showing VAS lengths in the morning, in the night, and in the next morning according to Example 1 of the present invention.

FIG. 4 is a graph showing respective concentrations of total amino acids in subjects' plasma in the morning, in the night, and in the next morning according to Example 1 of the present invention.

FIG. 5 is a graph showing respective concentrations of branched-chain amino acids in the subjects' plasma in the morning, in the night, and in the next morning according to Example 1 of the present invention.

FIG. 6 is a graph showing respective concentrations of aromatic amino acids in the subjects' plasma in the morning, in the night, and in the next morning according to Example 1 of the present invention.

FIG. 7 is a graph showing respective concentrations of cysteine in the subjects' plasma in the morning, in the night, and in the next morning according to Example 1 of the present invention.

FIG. 8 is a graph showing respective concentrations of methionine in the subjects' plasma in the morning, in the night, and in the next morning according to Example 1 of the present invention.

FIG. 9 is a graph showing respective concentrations of lysine in the subjects' plasma in the morning, in the night, and in the next morning according to Example 1 of the present invention.

FIG. 10 is a graph showing respective concentrations of arginine in the subjects' plasma in the morning, in the night, and in the next morning according to Example 1 of the present invention.

FIG. 11 is a graph showing respective concentrations of histidine in the subjects' plasma in the morning, in the night, and in the next morning according to Example 1 of the present invention.

FIG. 12 is a graph showing VAS lengths before fatigue loading and after four hours of fatigue loading according to Example 2 of the present invention.

FIG. 13 is a graph showing respective concentrations of valine in subjects' plasma before and after four hours of mental work according to Example 2 of the present invention.

FIG. 14 is a graph showing respective concentrations of leucine in the subjects' plasma before and after four hours of mental work according to Example 2 of the present invention.

FIG. 15 is a graph showing respective concentrations of isoleucine in the subjects' plasma before and after four hours of mental work according to Example 2 of the present invention.

FIG. 16 is a graph showing respective concentrations of glycine in the subjects' plasma before physical work and after four hours of mental work according to Example 2 of the present invention.

FIG. 17 is a graph showing respective concentrations of proline in the subjects' plasma before physical work and after four hours of mental work according to Example 2 of the present invention.

FIG. 18 is a graph showing respective concentrations of alanine in the subjects' plasma before physical work and after four hours of mental work according to Example 2 of the present invention.

FIG. 19 is a graph showing respective concentrations of asparagine in the subjects' plasma before physical work and after four hours of mental work according to Example 2 of the present invention.

FIG. 20 is a graph showing respective concentrations of lysine in the subjects' plasma before physical work and after four hours of mental work according to Example 2 of the present invention.

FIG. 21 is a graph showing respective concentrations of histidine in the subjects' plasma before physical work and after four hours of mental work according to Example 2 of the present invention.

FIG. 22 is a diagram showing a combined (mental and physical) fatigue loading method by using a driving simulator according to Example 3 of the present invention.

FIG. 23 is a graph showing respective concentrations of tryptophan in subjects' plasma before and after the driving simulator according to Example 3 of the present invention.

FIG. 24 is a diagram schematically showing a functional block of a fatigue evaluation apparatus according to the present embodiment.

### BEST MODE FOR CARRYING OUT THE INVENTION

In the following, a fatigue evaluation method according to the present invention, a kit therefor, and an application thereof will be described. Note that the present invention is not to be limited to this arrangement.

### (1) Fatigue Evaluation Method

The inventors have found that the degree of fatigue of a human being can be easily and quantitatively measured by measuring a concentration of amino acid in a body fluid taken from a subject. According to this method, there is no need for a huge and expensive apparatus and the body fluid can be taken from the subject in a short period of time, so that the subject is bound only for a short period of time and the method is easy for its user to carry out.

First, an outline of a fatigue evaluation method according the present invention will be described briefly. Note that the outline of the method described herein has much in common with outlines of a kit therefor and an application thereof described later.

In the foregoing method, first, a body fluid is taken from a subject so as to measure a concentration of amino acid in the subject's body fluid. The amino acid only needs to be a compound having a carboxyl group and an amino group in the same molecule but is preferably total amino acids, branched-chain amino acids, aromatic amino acids, cysteine, methionine, lysine, arginine, and histidine. Examples of the branched-amino acids include valine, leucine, and isoleucine. Examples of the aromatic amino acids include phenylalanine, tyrosine, and tryptophan. The body fluid only needs to be at least one type of body fluid selected from plasma, saliva, cerebrospinal fluid, and urine, but is preferably plasma.

Furthermore, the method for measuring the amino acid in the body fluid may be a conventional method and not to be limited in terms of techniques, conditions, and other features adopted therein. For example, the concentration of the amino acid in the body fluid is measured by liquid chromatography.

Further, the term “degree of fatigue” in the present invention means the degree of a debilitated physical or mental work ability which is caused by excessive physical and/or mental activity and which is accompanied by distinctive pathological discomfort and desire for relaxation. The term “debilitated physical or mental working ability” here means qualitatively or quantitatively degraded physical and mental working abilities.

As described above, the term “fatigue” in the present invention is categorized into physiological fatigue and pathological fatigue. “Physiological fatigue” is categorized into acute fatigue and chronic fatigue, and “acute fatigue” is categorized into mental fatigue, physical fatigue, and combined fatigue including both mental and physical elements. Meanwhile, “chronic fatigue” can be categorized in the same manner as “acute fatigue”. Further, it is preferable that a target for the degree of fatigue in the present invention be physiological fatigue, particularly acute fatigue. Furthermore, a target for the degree of fatigue in the present invention may be persistent fatigue.

The term “overwork” in the present invention means a state in which a continued state of physiological chronic fatigue collapses a biological rhythm, fatally devastates life-sustaining functioning, and finally causes pathological fatigue.

The term “mental fatigue” in the present invention means fatigue caused when emotional and mental activity such as patience, tension, or time-pressing fretfulness is excessively required in addition to psychological activity such as complex calculating, memorizing, or thinking.

The term “physical fatigue” in the present invention means fatigue caused by performing physical work.

The term “mental fatigue loading” in the present invention means giving mental fatigue including eye fatigue and mental stress.

The term “combined fatigue loading” in the present invention means giving combined fatigue including both physical and mental elements. Examples of the combined fatigue loading include driving of motor vehicles.

Further, in the fatigue evaluation method according to the present invention, it is determined that a lower concentration of amino acid in a body fluid is indicative of a higher degree of fatigue of a subject. This is a corollary of the fact that, as described later, a concentration of amino acid in a subject's body fluid decreases with an increase in the degree of fatigue of the subject.

Furthermore, the fatigue evaluation method according to the present invention can be carried out in whole or in part by using a conventional computing apparatus (information-processing apparatus) such as a computer. For example, in other words, the fatigue evaluation method according to the present invention includes: a taking-out step of taking out a body fluid from a subject; a measuring step of measuring a concentration of amino acid in the body fluid, and an evaluating step of evaluating the degree of fatigue of the subject in accordance with a result of measuring the concentration of the amino acid in the body fluid. The computing apparatus can be used especially in the evaluating step from among these steps.

In this specification, the present invention is mainly envisioned for a human being (subject). However, the present invention can be applied not only to the human being but also to various mammals such as laboratory animals. Particularly, animals such as a mouse, a rat, a rabbit, and a monkey are frequently used as laboratory animals. Therefore, application of the present invention to these living organisms is very useful especially in terms of development of health food products and pharmaceutical products.

### (2) Fatigue Evaluation Kit

In the following, a fatigue evaluation kit according to the present invention will be described. The fatigue evaluation kit according to the present invention is a kit for evaluating the degree of fatigue of a human being. That is, the fatigue evaluation kit according to the present invention only needs to be a kit for carrying out the fatigue evaluation method according to the present invention described in Section (1). More specifically, for example, the fatigue evaluation kit according to the present invention only needs to be a kit including: (a) means for taking out a body fluid from a subject; and (b) means for measuring a concentration of amino acid in the body fluid thus taken out. The means (b) may be means necessary to carry out a conventional measurement method. Specifically, examples of the means (b) include a reagent, an instrument, an apparatus, a catalyst, and other articles necessary to carry out the method described in Section (1) for measuring the concentration of the amino acid in the body fluid.

Furthermore, the fatigue evaluation kit according to the present invention may be kit including a conventional computing apparatus such as a computer.

### (3) Fatigue Evaluation Apparatus

In the foregoing embodiment, the present invention provides a fatigue evaluation apparatus for carrying out the fatigue evaluation method described in Section (1). The fatigue evaluation apparatus only needs to include at least: a member (means) for measuring a concentration of amino acid in a body fluid separated from a subject to be analyzed; and a member (means) for evaluating the degree of fatigue by using as an index the concentration of the amino acid. The fatigue evaluation apparatus may further include: a member (means) for visualizing an evaluation result and a member (means) for displaying an image, for example.

For example, FIG. 24 schematically shows a function block of the fatigue evaluation apparatus according to the present embodiment. As shown in FIG. 24, the fatigue evaluation apparatus 10 according to the present embodiment includes a measurement section 1, an evaluation section 2, a storage section 3, an input section 4, and an output section 5.

The measurement section 1 is not to be limited in terms of concrete arrangements and other features as long as it measures a concentration of amino acid in a body fluid of a living organism to be analyzed. For example, as described later in Examples, an arrangement in which measurement is carried out by using a conventional method such as a method for measuring a concentration of amino acid in a body fluid by liquid-chromatography, a commercially available amino acids concentration measurement kit, or other arrangements can be suitably used as the measurement section 1.

The evaluation section 2 is not to be limited in terms of concrete arrangements and other features as long as it evaluates, by using as an index a measurement result obtained by the measurement section 1, the degree of fatigue of an individual from which the body fluid has been taken. That is, the evaluation section 2 is a member for carrying out the foregoing fatigue evaluation method according to the present invention. As the evaluation section 2, a conventional computing apparatus can be used for example. An arrangement of the evaluation section 2 will be described in detail later.

The storage section 3 stores various types of information (general information such as a subject's name, sex, age, eating habit, and exercise habit evaluation and other types of information such as a type of body fluid used, a concentration of amino acid in a body fluid, and an evaluation result) used in the fatigue evaluation apparatus 10. Specifically, as the storage section 3, various conventional storage means may be suitably used such as semiconductor memories (e.g. RAM, ROM, and other semiconductor memories), magnetic disks (e.g. flexible disks, hard disks, and other magnetic disks), disks including optical disks (e.g. CD-ROM, MO, MD, DVD, and other optical disks), and cards (e.g. IC cards (including memory cards), optical cards, and other cards).

Further, the storage section 3 may be integrated with the fatigue evaluation apparatus 10 into one unit but may be a separate external storage device. Furthermore, both of the integrated storage section 3 and the external storage device may be provided. Examples of the integrated storage section 3 include a built-in hard disk, a built-in flexible disk drive, a built-in CD-ROM drive, or a built-in DVD-ROM drive. Examples of the external storage device include an external hard disk or external types of the various disk drives.

The input section 4 is not particularly limited as long as it makes it possible to input information regarding operation of the fatigue evaluation apparatus 1. As the input section 4, conventional input means such as a keyboard, a tablet, or a scanner can be suitably used.

The output section 5 is display means for displaying various types of information such as information and results, regarding operation of the fatigue evaluation apparatus 10, which includes a concentration of amino acid measured by the measurement section 1 and an evaluation result outputted by the evaluation section 2. Specifically, as the output section 5, various display devices such as conventional CRT displays and liquid crystal displays can be suitably used. However, the output section 5 is not to be limited to these displays.

Further, the output section 5 may record (print or imaging), on a recoding material such as a PPC sheet, various types of information that can be displayed on display means. Specifically, as the output section 5, a publicly known image formation device such as an inkjet printer or a laser printer can be suitably used. However, the output section 5 is not to be particularly limited to this image formation device. That is, the output section 5 is means for outputting various types of information in softcopy and/or means for outputting various types of information in hardcopy. Note that the output means used in the present invention are not to be limited to the display means and the printing means, but other output means may be provided.

In the following, functioning and operation of the evaluation section 2, which is a characteristic portion of the present invention, will be described in detail. For example, the evaluation section 2 determines that the degree of fatigue is high when a concentration of amino acid in a measurement result obtained by the measurement section 1 is lower than a predetermined value. The predetermined value here is, for example, a threshold value which can serve as an “index of fatigue” which has been obtained through an experiment using a plurality of subjects and a value of concentration of amino acid in a body fluid in a relaxed state (meaning the opposite of a fatigue state). The “predetermined value” can be stored in the storage section 3 to call up for each evaluation.

Further, the evaluation section 2 preferably determines that there is an overwork state due to accumulation of physiological acute fatigue developed in daily life, when the measurement result obtained by the measurement section 1 shows that the concentration of the amino acids is lower than the predetermined value. Further, it is preferable that a target for evaluation of the degree of fatigue be physiological acute fatigue developed in daily life, particularly mental fatigue.

As described above, the body fluid only needs to be at least one type of body fluid selected from a group consisting of plasma, saliva, cerebrospinal fluid, and urine, all of which have been separated from an individual organism. Further, the amino acid only needs to be at least one type of amino acid selected from a group consisting of total amino acids, branched-chain amino acids, aromatic amino acids, cysteine, methionine, lysine, arginine, and histidine.

Furthermore, it is preferable that the measurement section 1 measures respective concentrations of amino acid in a body fluid before and after a subject to be analyzed is subjected to fatigue loading, and it is preferable that the evaluation section 2 evaluate the degree of fatigue by using as an index a change in concentration of the amino acid in the body fluid between before and after the fatigue loading, on the basis of a measurement result obtained by the measurement section 1. That is, the evaluation section 2 determines that the subjects is fatigued either when the concentration of the amino acid in the body fluid before the fatigue loading is lower than the concentration of the amino acid in the body fluid after the fatigue loading or when the change in concentration of the amino acid in the body fluid between before and after the fatigue loading is lower than a predetermined value. The predetermined value here is, for example, a threshold value calculated with reference to a change in concentration of amino acid in a body fluid under no fatigue loading (in a relaxed state).

As described above, the fatigue evaluation apparatus according to the present embodiment makes it possible to easily and accurately carry out the foregoing fatigue evaluation method.

### (4) Application of the Present Invention

As described above, the fatigue evaluation apparatus, the fatigue evaluation method, and the fatigue evaluation kit according to the present invention make it possible to quantitatively measure and evaluate an anti-fatigue effect of an anti-fatigue substance in a subject's living organism by simply measuring respective concentrations of amino acid in the subject's body fluid before and after the subject takes the anti-fatigue substance. Furthermore, both of the method and the kit are easy to use and do not require a huge and expensive apparatus or long binding hours, so that both of the method and the kit have an advantage of being easy to handle for both a subject and a user.

Thus, the present invention also includes a method for measuring an anti-fatigue effect of an anti-fatigue substance, the method measuring the anti-fatigue effect of the anti-fatigue substance by using any one of the fatigue evaluation method and the fatigue evaluation kit according to the present invention. Further, in other words, the method for measuring an anti-fatigue effect of an anti-fatigue substance, for example, includes: (i) a before-intake measurement step of measuring a concentration of amino acid in a body fluid taken from a subject before the subject takes an anti-fatigue substance; (ii) an after-intake measurement step of measuring a concentration of amino acid in a body fluid taken from the subject after the subject has taken the anti-fatigue substance; (iii) a change-calculating step of calculating a change in concentration of the amino acid in the body fluid between before and after the intake of the anti-fatigue substance, on the basis of a result of measuring a change in concentration of the amino acid between before and after the intake of the anti-fatigue substance, the result being obtained by the before-intake measurement step and the after-intake measurement step; and (iv) an anti-fatigue effect measuring step of measuring an anti-fatigue effect in vivo of the anti-fatigue substance on the basis of the change in concentration of the amino acid in the body fluid between before and after the intake of the anti-fatigue substance, the change being obtained by the calculation step. Furthermore, the foregoing method may be performed in an experimental group (a group of subjects to whom the anti-fatigue substance is administered) and a control group (a group of subjects to whom no anti-fatigue substance is administered).

Note that the term “anti-fatigue” here means an effect of treating and inhibiting fatigue.

Further, the fatigue evaluation method and the fatigue evaluation kit according to the present invention can be applied for example to an anti-fatigue substance screening method. That is, the anti-fatigue substance screening method according to the present invention is not to be particularly limited in terms of concrete ways, conditions, and other features adopted therein as long as it is an anti-fatigue substance screening method performed by using either the fatigue evaluation method or the fatigue evaluation kit.

According to the forgoing screening method, for example, a subject is made to orally take a food group which may be used as an anti-fatigue food product, so that a food which actually exhibits an excellent anti-fatigue effect in vivo can be easily and objectively selected. Therefore, an anti-fatigue substance or an anti-fatigue food product obtained by the foregoing screening method is proved to be effective in vivo and therefore will be highly appreciated in the market.

Note that the anti-fatigue substance obtained by the foregoing screening method is also included in the present invention. That is, a novel anti-fatigue substance according to the present invention only needs to be obtained by the foregoing screening method.

Further, as fatigue is recognized as a social problem, a variety of anti-fatigue substances and anti-fatigue food products has become available in large quantities, and there has been a strong demand for development of a method for appropriately evaluating an anti-fatigue effect of each of these food products. Such a demand can be satisfied by the fatigue evaluation method, the fatigue evaluation kit, and the application thereof according to the present invention.

Exemplified as concrete examples of the application of the fatigue evaluation apparatus and the like according to the present invention are a method for evaluating an anti-fatigue effect of an anti-fatigue substance, an anti-fatigue effect evaluation system, and a method for screening a candidate substance for an anti-fatigue substance.

First, the method for evaluating an anti-fatigue effect of an anti-fatigue substance is not to be particularly limited in terms of concrete arrangements, instruments, conditions, and other features adopted therein as long as it includes the processes of: administering the anti-fatigue substance to a subject in a fatigue state; determining whether or not the subject has recovered from fatigue, by using a fatigue evaluation apparatus, a fatigue evaluation method, or a fatigue evaluation kit according to any one of Sections (1) to (3); and evaluating the anti-fatigue effect of the anti-fatigue substance by using as an index a degree to which the subjects have recovered from fatigue. According to the foregoing method, it is possible to easily and accurately evaluate an anti-fatigue effect of an anti-fatigue substance.

Further, the anti-fatigue effect evaluation system is not to be particularly limited in terms of concrete arrangements, and other features adopted therein as long as it includes: a first fatigue evaluation apparatus according to Section (1) for evaluating the degree of fatigue of a subject to whom an anti-fatigue substance has been administered; and a second fatigue evaluation apparatus for evaluating an anti-fatigue effect of the anti-fatigue substance by using as an index a degree to which the subject has recovered from fatigue, on the basis of an evaluation result obtained by the first fatigue evaluation apparatus. According to the anti-fatigue effect evaluation system, it is possible to easily and accurately evaluate an anti-fatigue effect of an anti-fatigue substance.

Further, the method for screening a candidate substance for an anti-fatigue substance is not to be particularly limited in terms of concrete arrangements, instruments, conditions, and other features adopted therein as long as it includes the processes of: administering a test article to a model animal in a fatigue state; determining whether or not the model animal has recovered from fatigue, by using a fatigue evaluation apparatus, a fatigue evaluation method, or a fatigue evaluation kit according to any one of Sections (1) to (3); and determining that the test article is a candidate substance for an anti-fatigue substance, by using as an index a degree to which the model animal has recovered from fatigue. According to the foregoing method, it is possible to easily and accurately screen an anti-fatigue substance.

Finally, each block of the fatigue evaluation apparatus 10, in particular, the measurement section 1 and the evaluation section 2 may be arranged according to hardware logic or may be achieved according to software by using an CPU as described below.

That is, the fatigue evaluation apparatus 10 for example includes a CPU (central processing unit) for executing a command from a control program for achieving. each function, a ROM (read only memory) for storing the control program, a RAM (random access memory) for extracting the control program, and a storage device (storage medium) such as memory for storing the control program and various types of data. An object of the present invention can be achieved also in the following manner. That is, a computer-readable storage medium storing program codes (an executable format program, an immediate code program, and a source program) of the control program of the fatigue evaluation apparatus 10 which control program is a software program for achieving the function is supplied to the fatigue evaluation apparatus 10, so that the computer (or CPU/MPU) reads out and executes the program codes stored in the storage medium.

Exemplified as the storage medium are tapes such as magnetic tapes and cassette tapes, disks including magnetic disks (e.g. floppy (registered trademark) disks and hard disks) and optical disks (e.g. CD-ROMs, MOs, MDs, DVDs, and CD-Rs), cards such as IC cards (including memory cards) and optical cards, or semiconductor memories (e.g. mask ROMs, EPROMs, EEPROMs, and flash ROMs).

Further, the fatigue evaluation apparatus 10 may be arranged so as to be connectable to a communications network so that the program codes are supplied through the communications network. The communications network is not to be particularly limited. As the communications network, for example, the Internet, an intranet, an extranet, a LAN, an ISDN, a VAN, a CATV communications network, a virtual private network, a telephone communications network, a mobile communications network, and a satellite communications network can be used. Further, a transmission medium used to form the communications network is not particularly limited. As the transmission medium, for example, wired media (e.g. an IEEE1394, a USB, a power line communication wire, a CATV line, a telephone line, an ADSL line) and wireless media (e.g. infrared (such as an IrDA and a remote controller), a Bluetooth (registered trademark), 802.11 wireless, an HDR, a mobile phone network, a satellite line, and a digitalized terrestrial network) can be used. Note that the present invention can be also achieved by the program codes in the form of a computer data signal embedded in a carrier wave which is embodied by electronic transmission.

In the following, examples will be described in conjunction with the accompanying drawings, and the embodiment of the present invention will be described further in detail. Needless to say, the present invention is not to be limited to the following examples, and details of the present invention may be varied in many ways. Furthermore, the present invention is not to be limited to the foregoing embodiment and can be varied in many ways within the scope of the following claims. Embodiments obtained by combining the technical means respectively disclosed in different embodiments are also included in the technical scope of the present invention.

The present invention is the fruits of “Research into the molecular and nervous system mechanisms for fatigue and feelings of fatigue, and into their prevention” through Special Coordination Funds for Promoting Science and Technology from the Ministry of Education, Science, Culture, Sports, Science and Technology of Japan.

### EXAMPLES

### Example 1

In the present example, experimentation was conducted in a relaxed state and a metal fatigue loading state. In either case, subjective fatigue sensation and a concentration of amino acid in plasma were measured at three points of time (in the morning of an experiment day, in the night of the experiment day, and in the next morning).

### [1] Fatigue Evaluation Method

[1-1] Subjects

Five healthy males and four healthy females served as subjects (with an average age of 27.6±5.5). Each of the subjects submitted a letter of consent to the experiment, and the experiment was approved by the Ethical Committee of the Kansai University of Welfare Sciences (Approval No. 1).

[1-2] Experimental Schedule

Table 1 is a schedule of the experiment conducted in the present example. The experimental schedule shows times when blood sample was taken from the subjects, times when the subjects were subjected to fatigue loading, times when the subjects were allowed to rest, and other times.

TABLE 1

| Time | Schedule |
|---|---|
| 8:30 | Gathering of subjects |
| 9:00 | VAS testing and blood sampling |
| 9:45 | Meal and rest |
| 10:15 | Fatigue loading: First term |
| 12:15 | VAS testing and blood sampling |
| 13:00 | Fatigue loading: Second term |
| 15:00 | VAS testing and blood sampling |
| 15:45 | Meal and rest |
| 16:15 | Fatigue loading: Third term |
| 18:15 | VAS testing and blood sampling |
| 19:00 | Fatigue loading: Fourth term |
| 21:00 | VAS testing and blood sampling |
| 21:45 | Meal |
| 23:00 | Bedtime |
| 6:30 | Wake-up |
| Next morning | |
| 7:00 | VAS testing and blood sampling |

[1-3] Mental Fatigue Loading Methods

Mental fatigue loading was carried out in a manner shown in Table 2.

TABLE 2

| | First Term | Second Term | Third Term | Fourth Term |
|---|---|---|---|---|
| ATMT | 45 min. | 45 min. | 45 min. | 45 min. |
| Character | 30 min. | 30 min. | 30 min. | 30 min. |
| Picking | | | | |
| Mirror Image | 45 min. | 45 min. | 45 min. | 45 min. |
| Copying | | | | |

[1-3-1] ATMT (Advanced Trail-Making Test)

The ATMT is originally used to evaluate an aging phenomenon and screen an early stage of dementia. The ATMT is a psychoneurotic tool expected to be used as a fatigue-measuring tool. The ATMT is also a visual search response test in which each subject is asked to quickly press target numbers of 1 to 25 shown on a touch-panel display. The ATMT is different from a TMT (Trail-Making Test), which has been conventionally conducted on a sheet of A4 paper (a test in which each subject is asked to connect target numbers of 1 to 25 with a single continuous line without lifting his/her pencil from the sheet). In the ATMT, a search response time can be measured for each of the target numbers. All the target numbers can be rearranged every time a response is done. The responded target number can be replaced by a new target number. For these reasons, the ATMT makes it possible to evaluate an increase in mental fatigue during execution of testing, a degree of utilization of working memory for raising search efficiency, and other variables. When a subject presses a target number from among target numbers of 1 to 25 shown on a touch panel of a personal computer, the target number is replaced by a new target number appearing in a given position (when the subject presses 1, 1 is replaced by 26; and when the subject presses 2, 2 is replaced by 27; and so on).

There are three patterns of arrangements of target numbers appearing on a screen. In Pattern A, pressing of a target button causes color-shifting of its number, so that the button is distinguished from other buttons. In Pattern B, pressing of a target button causes the button to disappear and causes another number to appear, so that 25 numbers are arranged on the screen. In Pattern C, pressing a target button causes its number to disappear but causes another number on the next screen to appear, so that 25 numbers are arranged at random every time a target button is pressed. When all the numbers have been pressed in the three patterns, the work is done and the duration of the work is calculated by a computer. All these patterns make one set.

In the present example, the existing ATMT was partially improved (by using 25 target numbers of 1 to 25) in order to apply the existing ATMT to mental work loading. Test A, Test B, and Test C were continuously repeated for a predetermined period of time shown in Table 2.

[1-3-2] Kana Pick-up Test

This is a mental fatigue loading method in which each subject is asked to continue for 25 minutes to circle vowels (five vowels consisting of “a”, “e”, “i”, “o”, and “u”) which he/she finds in sentences of a predetermined book and then is asked to answer simple questions for five minutes about the sentences which he/she has read.

[1-3-3] Mirror Drawing Test

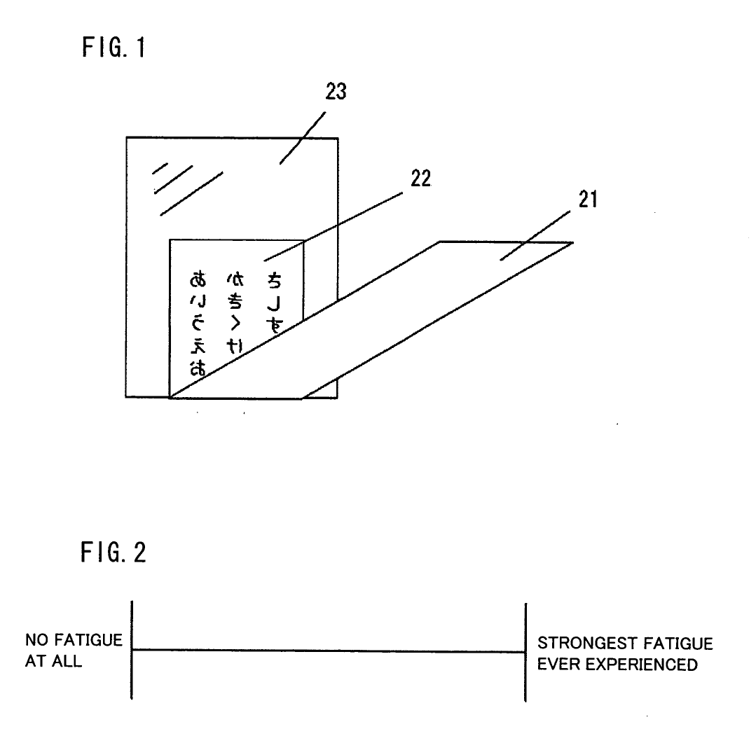

This is a mental fatigue loading method in which each subject is asked to continue for a predetermined period of time shown in Table 2 to directly copy onto a sheet at hand a mirror reversed state of characters reflected on a mirror (see FIG. 1).

[1-4] VAS Testing

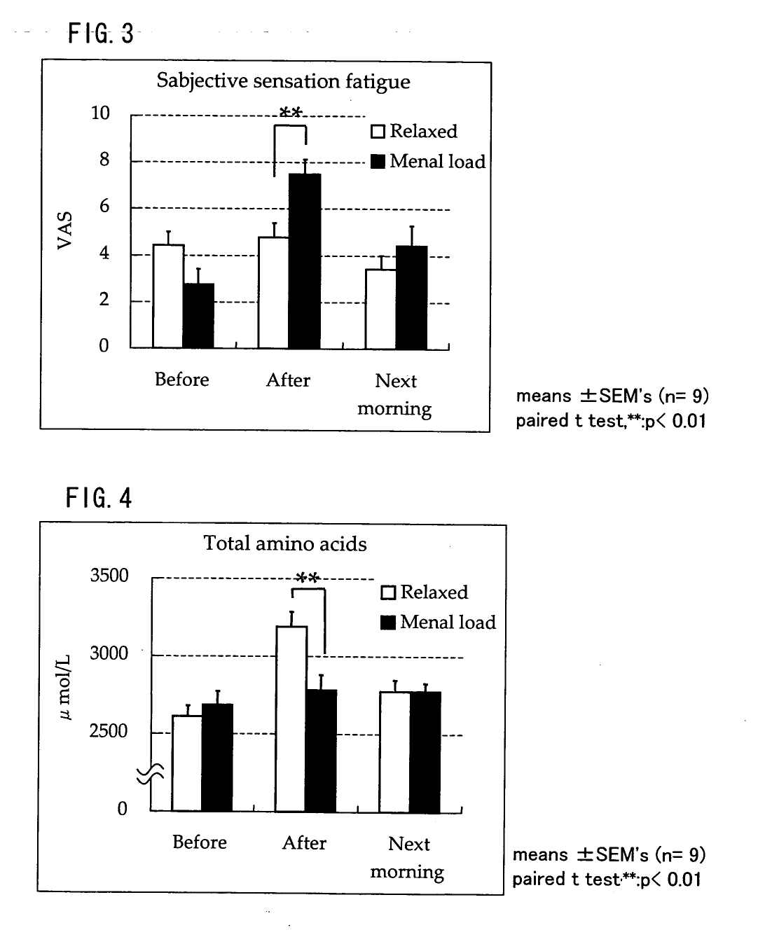

The VAS testing is an evaluation method in which each subject is shown a line segment written on a sheet with expressions serving as criteria for a target variable at both ends of the line segment, and then asked to mark on the line segment where the target variable lies. An advantage of the method is that a quantitative answer to a question about the target variable is obtained by measuring how far the target variable is from the left end of the line segment, so that answers obtained from many people can be averaged out. FIG. 2 shows a VAS test paper used in the present example (FIG. 3 shows a result of the VAS testing).

[1-5] Measurement of Concentrations of Amino Acids In Plasma

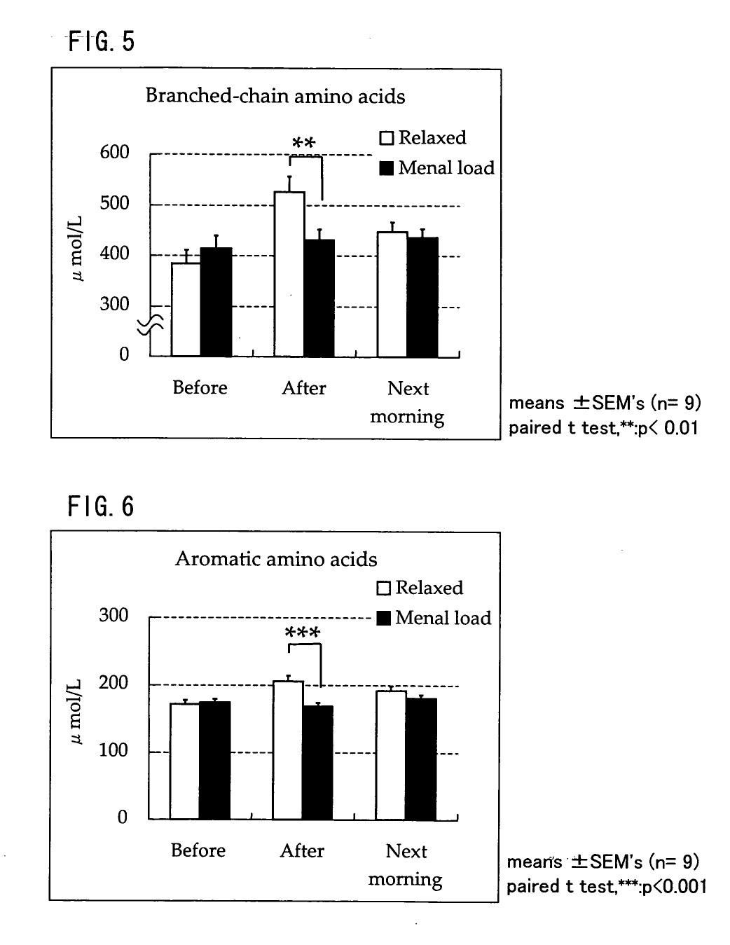

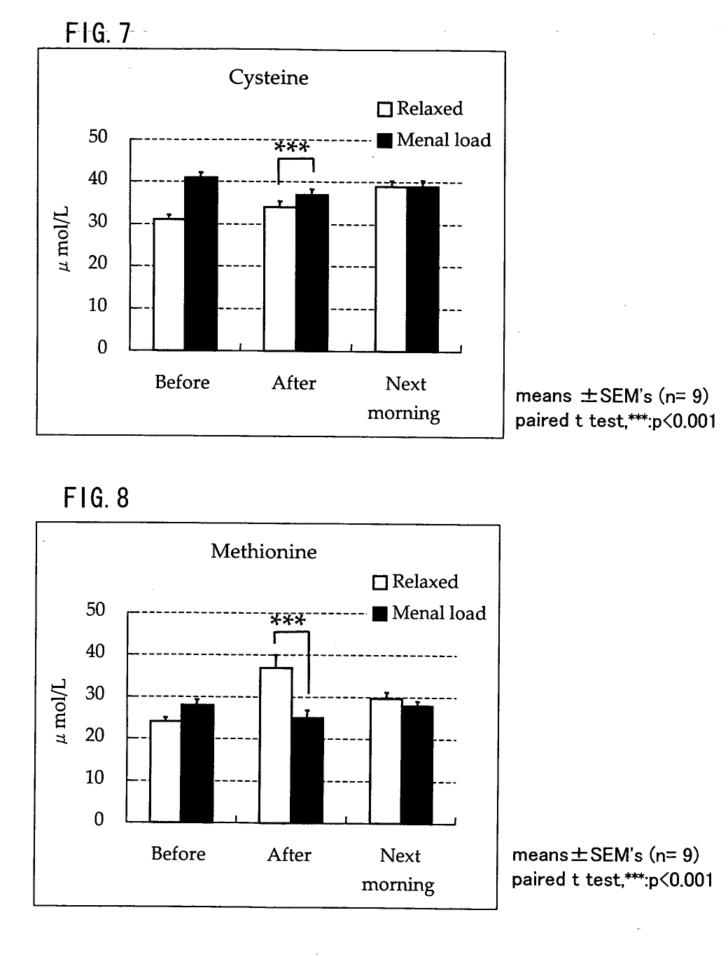

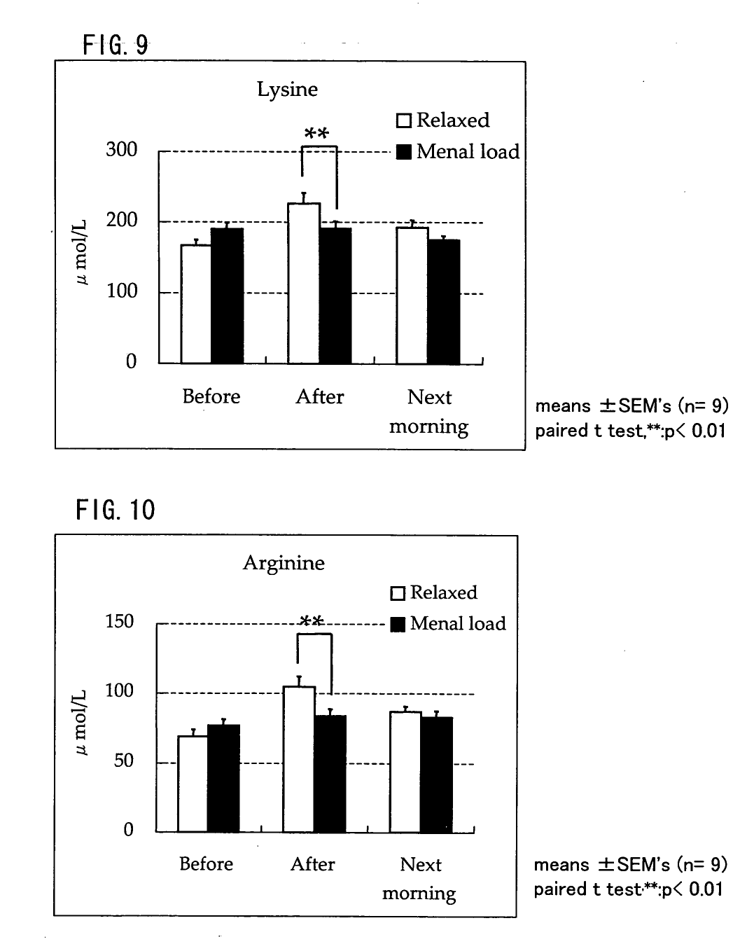

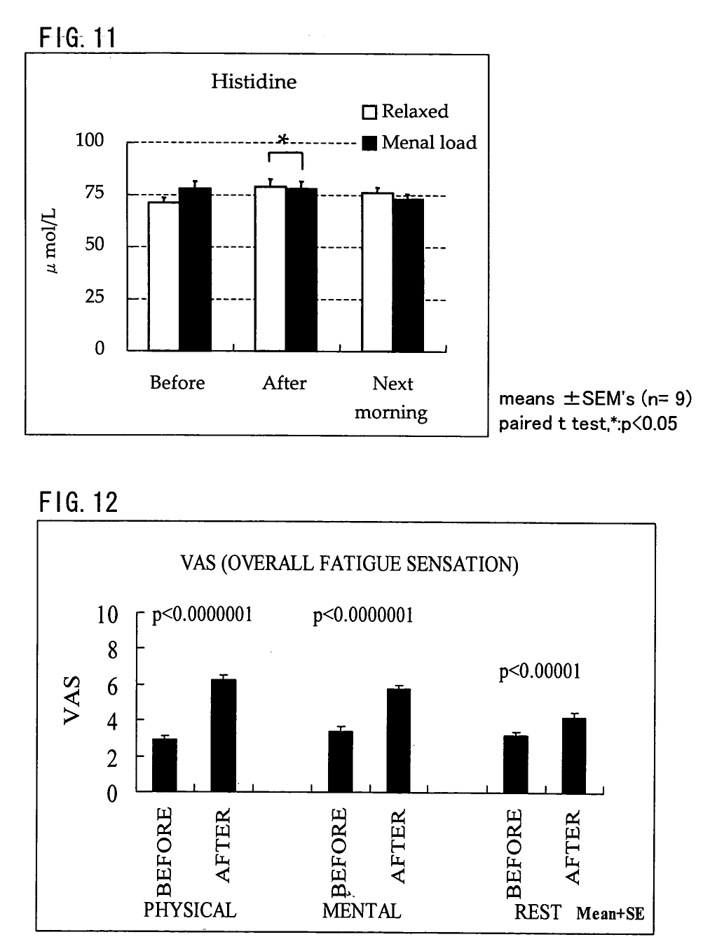

Plasma was taken from the subjects according to the experimental schedule shown in Table 1 so as to measure concentrations of amino acids in their plasma. FIG. 4 shows a result of measuring concentrations of total amino acids. FIG. 5 shows a result of measuring concentrations of branched-chain amino acids. FIG. 6 shows a result of measuring concentrations of aromatic amino acids. FIG. 7 shows a result of measuring concentrations of cysteine. FIG. 8 shows a result of measuring concentrations of methionine. FIG. 9 shows a result of measuring concentrations of lysine. FIG. 10 shows a result of measuring concentrations of arginine. FIG. 11 shows a result of measuring concentrations of histidine.

### [2] Results

Cortisol in saliva normally increases due to stress and exercise stress, and there was a significant decrease in cortisol in saliva in the mental fatigue loading state as compared with the relaxed state (data not shown). This shows that the mental fatigue loading shown in [1] is different from normal stress and exercise stress.

[2-1] VAS Testing

Lengths of VAS line segments were measured, and there was a significant difference between the lengths in the relaxed state and the lengths in the mental fatigue loading state. The lengths in the relaxed state were 4.42 (cm) in the morning and 4.78 (cm) in the night. The lengths in the mental fatigue loading state were 2.75 (cm) in the morning and 7.49 (cm) in the night. Whereas a change (Night-Morning) in the relaxed state was +0.36 (cm), a change (Night-Morning) in the mental fatigue loading state was +4.74 (cm). Thus, it was confirmed that the mental fatigue loading had raised the degree of fatigue.

[2-2] Measurement of Concentrations of Total Amino Acids In Plasma

Concentrations of total amino acids in plasma in the relaxed state were 2613 (μmol/L) in the morning and 3189 (μmol/L) in the night. Concentrations of total amino acids in plasma in the mental fatigue loading state were 2685 (μmol/L) in the morning and 2782 (μmol/L) in the night. Whereas a change (Night-Morning) in the relaxed state was +576 (μmol/L), a change (Night-Morning) in the mental fatigue loading state was +97 (μmol/L). This showed that there was a relative decrease of 479 (μmol/L). Since it was confirmed according to the testing of [1-4] that the mental fatigue loading had raised the degree of fatigue of the subjects, it became clear that it is possible to evaluate a decrease in concentration of total amino acids in a subject's plasma as being indicative that the subject has a high degree of fatigue.

[2-3] Measurement of Concentrations of Branched-Chain Amino Acids In Plasma

Concentrations of branched-chain amino acids in plasma in the relaxed state were 384 (μmol/L) in the morning and 526 (μmol/L) in the night. Concentrations of branched-chain amino acids in plasma in the mental fatigue loading state were 414 (μmol/L) in the morning and 431 (μmol/L) in the night. Whereas a change (Night-Morning) in the relaxed state was +142 (μmol/L), a change (Night-Morning) in the mental fatigue loading state was +17 (μmol/L). This showed that there was a relative decrease of 125 (μmol/L). Since it was confirmed according to the testing of [1-4] that the mental fatigue loading had raised the degree of fatigue of the subjects, it became clear that it is possible to evaluate a decrease in concentrations of branched-chain amino acids in a subject's plasma as being indicative that the subject has a high degree of fatigue.

[2-4] Measurement of Concentrations of Aromatic Amino Acids In Plasma Concentrations of aromatic amino acids in plasma in the relaxed state were 171 (μmol/L) in the morning and 206 (μmol/L) in the night. Concentrations of aromatic amino acids in plasma in the mental fatigue loading state were 174 (μmol/L) in the morning and 169 (μmol/L) in the night. Whereas a change (Night-Morning) in the relaxed state was +35 (μmol/L), a change (Night-Morning) in the mental fatigue loading state was -5 (μmol/L). This showed that there was a relative decrease of 40 (μmol/L). Since it was confirmed according to the testing of [1-4] that the mental fatigue loading had raised the degree of fatigue of the subjects, it became clear that it is possible to evaluate a decrease in concentration of aromatic amino acids in a subject's plasma as being indicative that the subject has a high degree of fatigue.

[2-5] Measurement of Concentrations of Cysteine In Plasma

Concentrations of cysteine in plasma in the relaxed state were 31 (μmol/L) in the morning and 34 (μmol/L) in the night. Concentrations of cysteine in plasma in the mental fatigue loading state were 41 (μmol/L) in the morning and 37 (μmol/L) in the night. Whereas a change (Night-Morning) in the relaxed state was +3 (μmol/L), a change (Night-Morning) in the mental fatigue loading state was -4 (μmol/L). This showed that there was a relative decrease of 7 (μmol/L). Since it was confirmed according to the testing of [1-4] that the mental fatigue loading had raised the degree of fatigue of the subjects, it became clear that it is possible to evaluate a decrease in concentration of cysteine in a subject's plasma as being indicative that the subject has a high degree of fatigue.

[2-6] Measurement of Concentrations of Methionine In Plasma

Concentrations of methionine in plasma in the relaxed state were 24 (μmol/L) in the morning and 37 (μmol/L) in the night. Concentrations of methionine in plasma in the mental fatigue loading state were 28 (μmol/L) in the morning and 25 (μmol/L) in the night. Whereas a change (Night-Morning) in the relaxed state was +13 (μmol/L), a change (Night-Morning) in the mental fatigue loading state was -3 (μmol/L). This showed that there was a relative decrease of 16 (μmol/L). Since it was confirmed according to the testing of [1-4] that the mental fatigue loading had raised the degree of fatigue of the subjects, it became clear that it is possible to evaluate a decrease in concentration of methionine in a subject's plasma as being indicative that the subject has a high degree of fatigue.

[2-7] Measurement of Concentrations of Lysine In Plasma

Concentrations of lysine in plasma in the relaxed state were 167 (μmol/L) in the morning and 226 (μmol/L) in the night. Concentrations of lysine in plasma in the mental fatigue loading state were 190 (μmol/L) in the morning and 191 (μmol/L) in the night. Whereas a change (Night-Morning) in the relaxed state was +59 (μmol/L), a change (Night-Morning) in the mental fatigue loading state was +1 (μmol/L). This showed that there was a relative decrease of 58 (μmol/L). Since it was confirmed according to the testing of [1-4] that the mental fatigue loading had raised the degree of fatigue of the subjects, it became clear that it is possible to evaluate a decrease in concentration of lysine in a subject's plasma as being indicative that the subject has a high degree of fatigue.

[2-8] Measurement of Concentrations of Arginine In Plasma

Concentrations of arginine in plasma in the relaxed state were 69 (μmol/L) in the morning and 105 (μmol/L) in the night. Concentrations of arginine in plasma in the mental fatigue loading state were 77 (μmol/L) in the morning and 84 (μmol/L) in the night. Whereas a change (Night-Morning) in the relaxed state was +36 (μmol/L), a change (Night-Morning) in the mental fatigue loading state was +7 (μmol/L). This showed that there was a relative decrease of 29 (μmol/L). Since it was confirmed according to the testing of [1-4] that the mental fatigue loading had raised the degree of fatigue of the subjects, it became clear that it is possible to evaluate a decrease in concentration of arginine in a subject's plasma as indicative that the subject has a high degree of fatigue.

[2-9] Measurement of Concentrations of Histidine In Plasma

Concentrations of histidine in plasma in the relaxed state were 71 (μmol/L) in the morning and 79 (μmol/L) in the night. Concentrations of histidine in plasma in the mental fatigue loading state were 78 (μmol/L) in the morning and 78 (μmol/L) in the night. Whereas a change (Night-Morning) in the relaxed state was +8 (μmol/L), a change (Night-Morning) in the mental fatigue loading state was 0 (μmol/L). This showed that there was a relative decrease of 8 (μmol/L). Since it was confirmed according to the testing of [1-4] that the mental fatigue loading had raised the degree of fatigue of the subjects, it became clear that it is possible to evaluate a decrease in concentration of histidine in a subject's plasma as indicative that the subject has a high degree of fatigue.

### Example 2

### [3] Fatigue Evaluation Method 2

[3-1] Subjects

Twenty-three healthy males and twenty-four healthy females served as subjects (with an average age of 39.9±11.1). The experiment was conducted under approval of the Joint Committee of Inquiry (led by Mr. Masaharu Inoue, an attorney) of Soiken and Soiken Clinic. In compliance with the spirit of Helsinki Declaration (adopted in 1964; amended in 1975, 1983, 1989, 1996, and 2000), each of the subjects had a doctor's sufficient explanation of the research, the methodology, and other features before submitting a letter of consent to the experiment.

[3-2] Experimental Schedule

Table 3 is a schedule of the experiment conducted in the present example. The experimental schedule shows times when plasma was taken from the subjects, times when the subjects were subjected to fatigue loading, times when the subjects were allowed to rest, and other times.

TABLE 3

| | | |
|---|---|---|
| Time | Events | Measured Characteristics |

The day before the loading day

| | | |
|---|---|---|
| 15:00 | Check in at hotel | |
| 15:30 | Gather at lobby and move to | |
| | experimental site | |
| 16:00 | Explain experiment and work | Explain outline of experiment and |
| | and tour around site | practice work |
| 18:00 | Move to hotel | |
| 18:30 | Arrive at hotel | |
| 19:00 | Dinner at restaurant of hotel | |
| | (same menu each time) | |
| 20:30 | Check quantity of food intake | |
| | and give instructions on stay | |
| | at hotel | |
| 21:00 | Bedtime in single room of | |
| | hotel | |

The loading day

| | | |
|---|---|---|
| 6:30 | Wake-up | |
| 7:00 | Gather at lobby and move to test site | |
| 7:30 | Arrive at test site | |
| 7:35 | Explain experimental procedure | |
| | briefly | |
| 7:40 | First measurement (before loading) | (i) Emotion (face scale); line segment (VAS); fatigue scale |
| | | (fatigue questionnaire); blood pressure, pulse, and body |
| | | temperature; and blood sampling. *Intake of sugar after |
| | | blood sampling |
| 8:25 | Start loading experiment: First term | |
| | (mental work, physical work, and no | |
| | load) | |
| 9:55 | Second measurement (after two-hour | (ii) Emotion (face scale); line segment (VAS), blood pressure, |
| | loading) | pulse, and body temperature and blood sampling |
| 10:30 | Fatigue loading experiment: Second | |
| | term (mental work, physical work, | |
| | and no load) | |
| 12:00 | Third measurement (immediately | (iii) Emotion (face scale); line segment (VAS); fatigue scale |
| | after four hours of loading) | (fatigue questionnaire); blood pressure, pulse, and body |
| | | temperature; and blood sampling |
| 12:50 | Lunch (rice ball) (same menu each | |
| | time), End of experiment | |

[3-3] Experimental Design

The experiment was conducted across three groups: a no-load control group, a mental workload group, and a physical workload group.

[3-4] Fatigue Loading Methods

Two types of fatigue loading were carried out: (i) mental fatigue loading and (ii) physical fatigue loading.

[3-4-1] Methods for Loading Fatigue by Mental Work

Three types of mental fatigue loading method were adopted: a character-picking test, an ATMT, and an Uchida-Kraepelin Test. According to the experimental schedule, mental fatigue loading work occurred in two terms. Each of the two terms lasted two hours and included the character-picking test (30 minutes), the ATMT (45 minutes), and the Uchida-Kraepelin Test (30 minutes) in this order.

(a) Character-Picking Test

The character-picking test was conducted in the same manner as in Example 1. In this test, each of the subjects was subjected to mental work loading for 30 minutes without a break.

(b) ATMT (Advanced Trail-Making Test)

The ATMT was conducted in the same manner as in Example 1. In this test, each of the subjects was subjected to mental work loading for approximately 30 minutes without a break.

[3-4-2] Method For Loading Fatigue By Physical Work

Each of the subjects was subjected to physical work loading by pedaling an ergometer according to the experimental schedule. The exercise intensity was set to WattAT80%, where 80% of heart rate at an AT (anaerobic threshold) is obtained. The day before the experiment, the exercise intensity was calculated by measuring the subject's VO₂ and heart rate at the AT by using an ergometer (Aerobike 75XL ME manufactured by Combi Corporation) and a respiratory metabolism measurement system (Aeromonitor AE-300S manufactured by Minato Medical Science Co., Ltd.). On the experiment day, physical work loading occurred in two two-hour-long terms with an exercise intensity of WattAT80%.

[3-5] VAS Testing

The VAS testing was conducted in the same manner as in Example 1 according to the experimental schedule. FIG. 12 shows a result of the VAS testing.

[3-6] Measurement of Concentrations of Amino Acids In Plasma

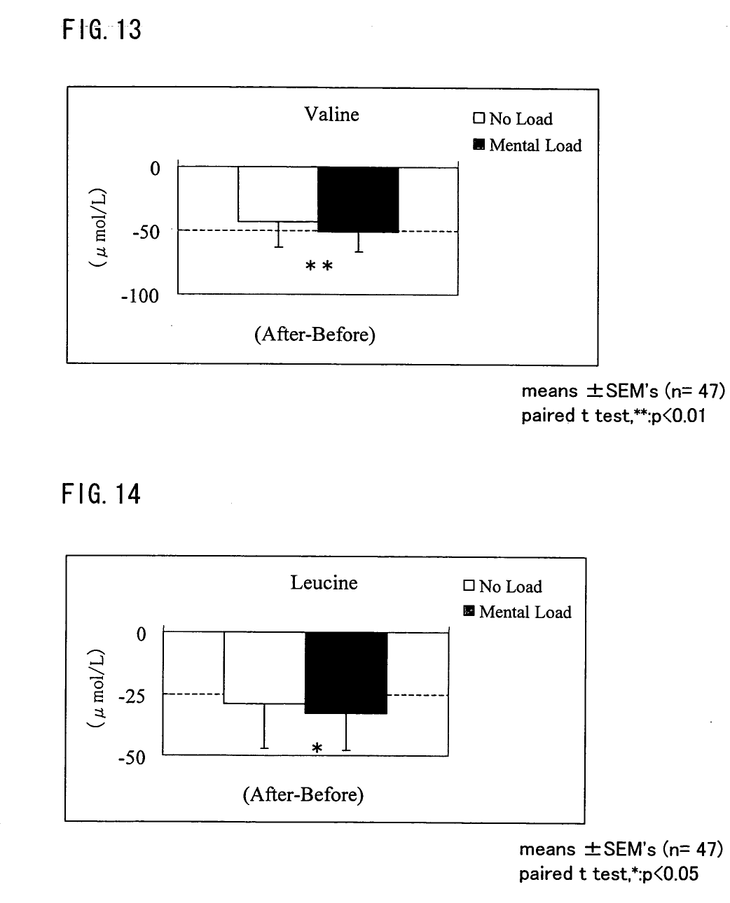

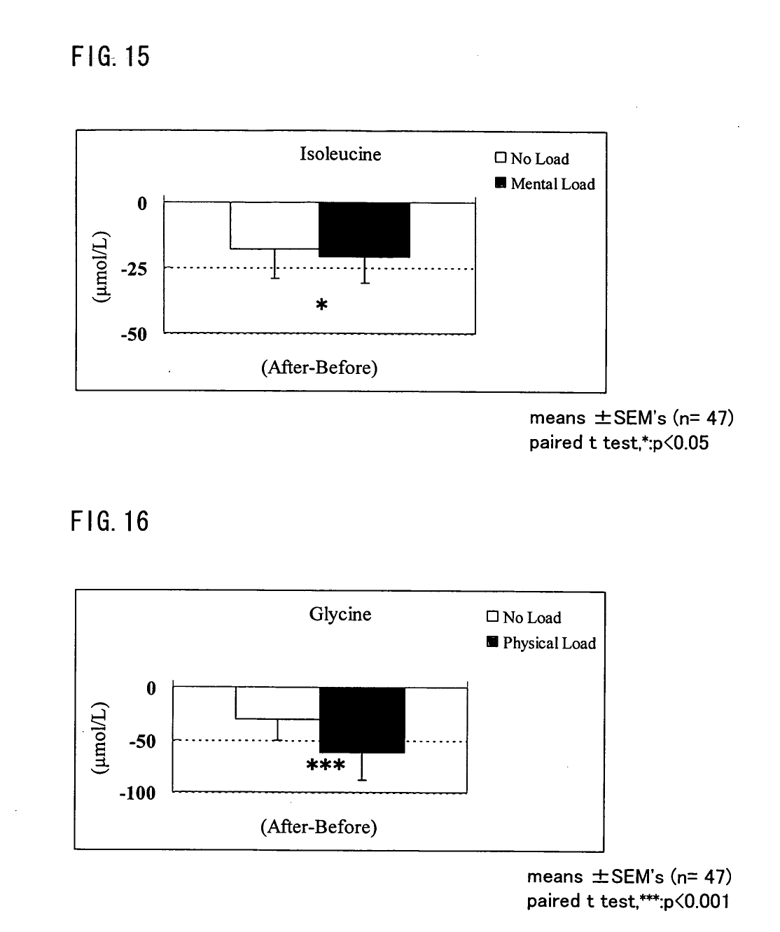

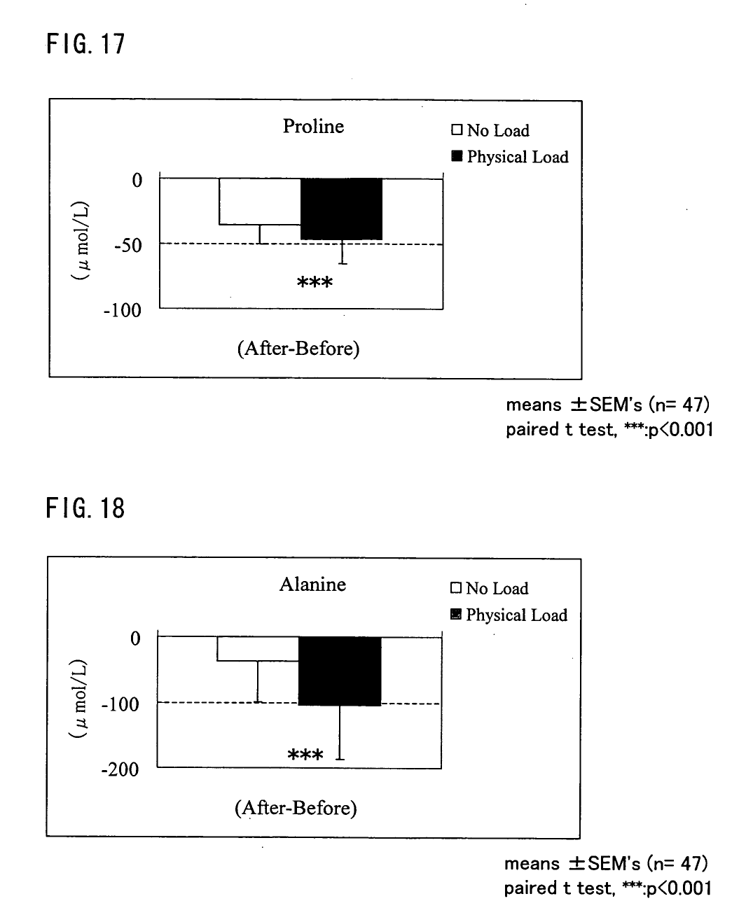

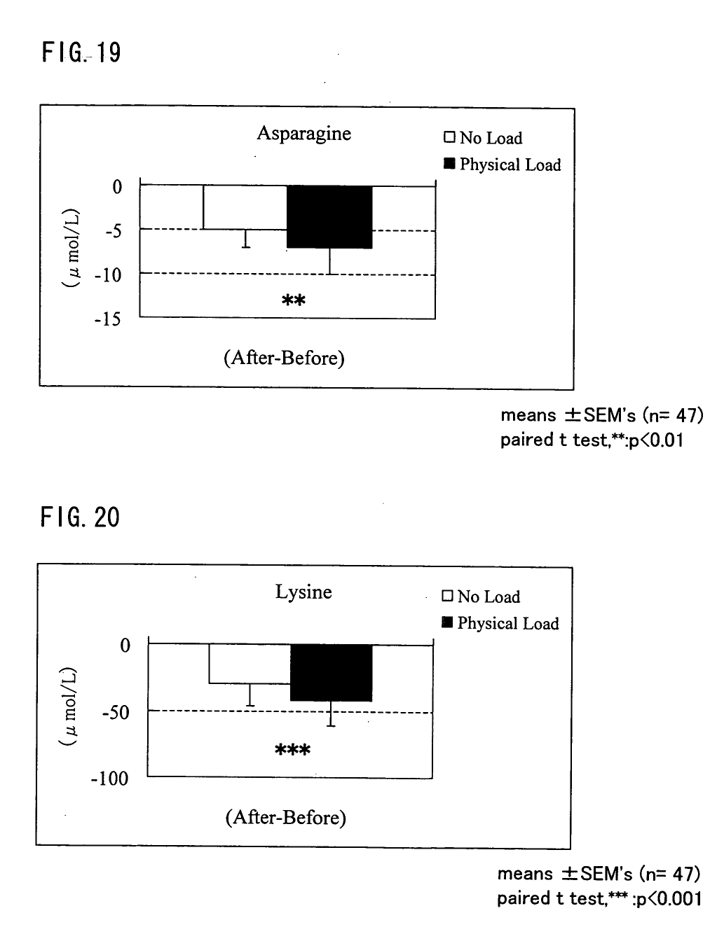

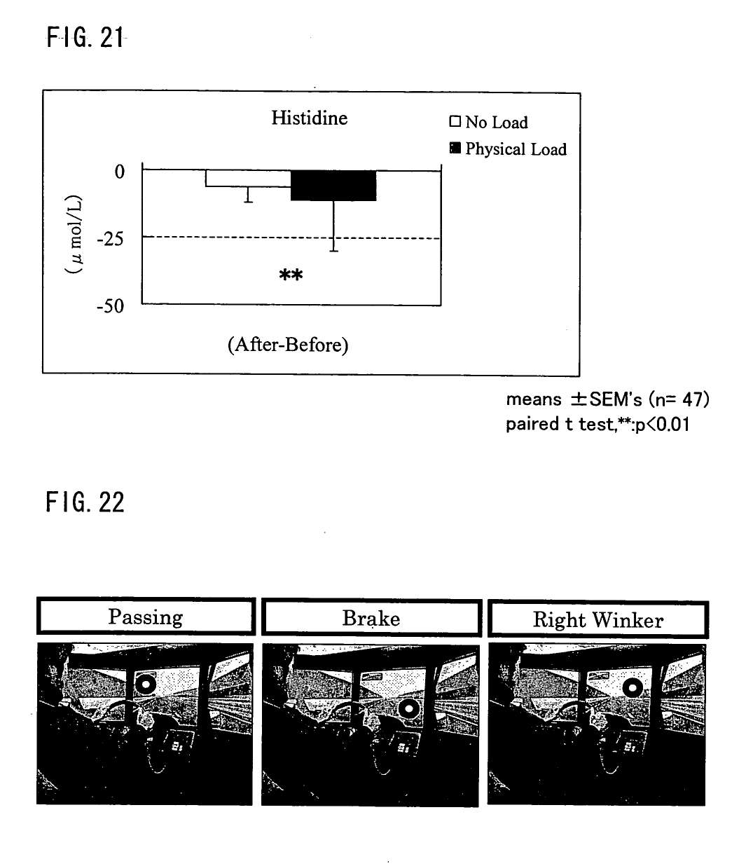

Plasma was taken from the subjects according to the experimental schedule shown in Table 3 so as to measure concentrations of amino acids in their plasma. FIG. 13 shows a result of measuring concentrations of valine, which is one type of branched-chain amino acid, in the mental workload group. FIG. 14 shows a result of measuring concentrations of leucine, which is another type of branched-chain amino acid, in the mental workload group. FIG. 15 shows a result of measuring concentrations of isoleucine, which is another type of branched-chain amino acid, in the metal workload group. FIG. 16 shows a result of measuring concentrations of glycine in the physical workload group. FIG. 17 shows a result of measuring concentrations of proline in the physical workload group. FIG. 18 shows a result of measuring concentrations of alanine in the physical workload group. FIG. 19 shows a result of measuring concentrations of asparagine in the physical workload group. FIG. 20 shows a result of measuring concentrations of lysine in the physical workload group. FIG. 21 shows a result of measuring concentrations of histidine in the physical workload group.

### [4] Results

[4-1] VAS Testing

In both of the mental workload group and the physical workload group, changes in VAS evaluation of fatigue sensation during four hours of fatigue loading were significantly larger than a change in the no-load group. Since the subjects in the two workload groups developed their subjective fatigue, it was confirmed that the subjects were fatigued by the fatigue loading methods.

[4-2] Changes In Concentration of Amino Acids In Plasma

[4-2-1] A Change In Concentration of Valine In Plasma Under Mental Work Loading

In the no-load group, a change (decrease) in concentration of valine in plasma between before and after four hours of fatigue loading was −43±20 (μmol/L). In the mental workload group, a change in concentration of valine in plasma between before and after four hours of fatigue loading was −51±15 (μmol/L). This meant that mental work had caused valine in plasma to decrease significantly. Since it was confirmed according to the testing of [3-5] that the metal fatigue loading had raised the degree of fatigue of the subjects, it became clear that it is possible to evaluate a wide change in concentration of valine in a subject's plasma as being indicative that the subject has a high degree of fatigue.

[4-2-2] A Change In Concentration of Leucine In Plasma Under Mental Work Loading

In the no-load group, a change (decrease) in concentration of leucine in plasma between before and after four hours of fatigue loading was −29±18 (μmol/L). In the mental workload group, a change in concentration of leucine in plasma between before and after four hours of fatigue loading was −33±16 (μmol/L). This meant that mental work had caused leucine in plasma to decrease significantly. Since it was confirmed according to the testing of [3-5] that the metal fatigue loading had raised the degree of fatigue of the subjects, it became clear that it is possible to evaluate a wide change in concentration of leucine in a subject's plasma as being indicative that the subject has a high degree of fatigue.

[4-2-3] A Change In Concentration of Isoleucine In Plasma Under Mental Work Loading

In the no-load group, a change (decrease) in concentration of isoleucine in plasma between before and after four hours of fatigue loading was −18±11 (μmol/L). In the mental workload group, a change in concentration of isoleucine in plasma between before and after four hours of fatigue loading was −21±10 (μmol/L). This meant that mental work had caused isoleucine in plasma to decrease significantly. Since it was confirmed according to the testing of [3-5] that the metal fatigue loading had raised the degree of fatigue of the subjects, it became clear that it is possible to evaluate a wide change in concentration of isoleucine in a subject's plasma as being indicative that the subject has a high degree of fatigue.

[4-2-4] A Change In Concentration of Glycine In Plasma Under Physical Work Loading

In the no-load group, a change (decrease) in concentration of glycine in plasma between before and after four hours of fatigue loading was −30±21 (μmol/L). In the physical workload group, a change in concentration of glycine in plasma between before and after four hours of fatigue loading was −62±26 (μmol/L). This meant that physical work had caused glycine in plasma to decrease significantly. Since it was confirmed according to the testing of [3-5] that the fatigue loading had raised the degree of fatigue of the subjects, it became clear that it is possible to evaluate a wide change in concentration of glycine in a subject's plasma as being indicative that the subject has a high degree of fatigue.

[4-2-5] A Change In Concentration of Proline In Plasma Under Physical Work Loading

In the no-load group, a change (decrease) in concentration of proline in plasma between before and after four hours of fatigue loading was −36±14 (μmol/L). In the physical workload group, a change in concentration of proline in plasma between before and after four hours of fatigue loading was −47±18 (μmol/L). This meant that physical work had caused proline in plasma to decrease significantly. Since it was confirmed according to the testing of [3-5] that the fatigue loading had raised the degree of fatigue of the subjects, it became clear that it is possible to evaluate a wide change in concentration of proline in a subject's plasma as being indicative that the subject has a high degree of fatigue.

[4-2-6] A Change In Concentration of Alanine In Plasma Under Physical Work Loading

In the no-load group, a change (decrease) in concentration of alanine in plasma between before and after four hours of fatigue loading was −37±62 (μmol/L). In the physical workload group, a change in concentration of alanine in plasma between before and after four hours of fatigue loading was −105±82 (μmol/L). This meant that physical work had caused alanine in plasma to decrease significantly. Since it was confirmed according to the testing of [3-5] that the fatigue loading had raised the degree of fatigue of the subjects, it became clear that it is possible to evaluate a wide change in concentration of alanine in a subject's plasma as being indicative that the subject has a high degree of fatigue.

[4-2-7] A Change In Concentration of Asparagine In Plasma Under Physical Work Loading

In the no-load group, a change (decrease) in concentration of asparagine in plasma between before and after four hours of fatigue loading was −5±2 (μmol/L). In the physical workload group, a change in concentration of asparagine in plasma between before and after four hours of fatigue loading was −7±3 (μmol/L). This meant that physical work had caused asparagine in plasma to decrease significantly. Since it was confirmed according to the testing of [3-5] that the fatigue loading had raised the degree of fatigue of the subjects, it became clear that it is possible to evaluate a wide change in concentration of asparagine in a subject's plasma as being indicative that the subject has a high degree of fatigue.

[4-2-8] A Change In Concentration of Lysine In Plasma Under Physical Work Loading

In the no-load group, a change (decrease) in concentration of lysine in plasma between before and after four hours of fatigue loading was −30±16 (μmol/L). In the physical workload group, a change in concentration of lysine in plasma between before and after four hours of fatigue loading was −42±19 (μmol/L). This meant that physical work had caused lysine in plasma to decrease significantly. Since it was confirmed according to the testing of [3-5] that the fatigue loading had raised the degree of fatigue of the subjects, it became clear that it is possible to evaluate a wide change in concentration of lysine in a subject's plasma as being indicative that the subject has a high degree of fatigue.

[4-2-9] A Change In Concentration of Histidine In Plasma Under Physical Work Loading

In the no-load group, a change (decrease) in concentration of histidine in plasma between before and after four hours of fatigue loading was −6±6 (μmol/L). In the physical workload group, a change in concentration of histidine in plasma between before and after four hours of fatigue loading was −11±9 (μmol/L). This meant that physical work had caused histidine in plasma to decrease significantly. Since it was confirmed according to the testing of [3-5] that the fatigue loading had raised the degree of fatigue of the subjects, it became clear that it is possible to evaluate a wide change in concentration of histidine in a subject's plasma as being indicative that the subject has a high degree of fatigue.

### Example 3

### [5] Fatigue Evaluation Method 3

[5-1] Subjects

Twelve healthy males who were in their twenties served as subjects. Each of the subjects was right-handed, had at least one year of experience as a licensed driver, and drove an ordinary motor vehicle at least once a week. Further, patients with sinus problems, smokers, users of drugs (such as caffeinated or spiced food products and anti-allergic drugs, which affect a central nervous system) were excluded.

[5-2] Experimental Schedule

Table 4 is a schedule of the experiment conducted in the present example. The experimental schedule shows times when plasma was taken from the subjects, times when the subjects were subjected to fatigue loading, times when the subjects were allowed to rest, and other times.

TABLE 4

| | |
|---|---|
| Time | Schedule |

Day before the experiment day

| | |
|---|---|
| 19:00 | Gathering of subjects |
| 19:30 | Practice of simulator driving |
| 20:00 | Meal |

Experiment day

| | |
|---|---|
| 9:00 | Gathering of subjects |
| 9:10 | Blood sampling and blood test |
| 9:40 | Meal and intake of tryptophan or placebo |
| 10:00 | Ten minutes of simulator driving |
| 10:10 | Four hours of fatigue loading |
| 14:10 | Ten minutes of simulator driving |
| 14:20 | Blood sampling and blood test |

[5-3] Method For Loading Complex Fatigue

The subject was subjected to driving work loading by using a driving simulator (ACCESS MASTER AM2330 manufactured by Tasknet Inc.).

In order to study an effect of tryptophan on fatigue, each of the subjects in a tryptophan group who received tryptophan before loading was subjected to four hours of simulator driving without a break, and each of the subjects in a placebo group who received a placebo before loading was subjected to four hours of simulator driving without a break. The same subject received 5 mg/kg of tryptophan or a placebo at least one week apart, and participated in the experiment.

The simulator driving assumed driving of an ordinary motor vehicle on a highway. An upper speed limit was set at 120 km/h, and the subject was prohibited from overtaking and dangerous driving. The subject was instructed to step on a brake as soon as possible when a green dot was shown on a screen. The subject was instructed to give a passing signal as soon as possible when a red dot was shown on the screen. The subject was instructed to give a right-turn signal as soon as possible when a yellow dot was shown on the screen. The simulator driving was set so that any one of the dots (stimuli) was shown once per minute on average. Efficiency of the subject's work during driving was evaluated by using as an index reaction times. The reaction times are intervals between the onset of stimuli and the responses. which the subject makes by stepping on the brake (Brake), giving the passing signal (Passing), and giving the right-turn signal (Right Winker) (FIG. 22).

[5-4] A Change In Concentration of Amino Acids In Plasma Under Complex Fatigue Loading

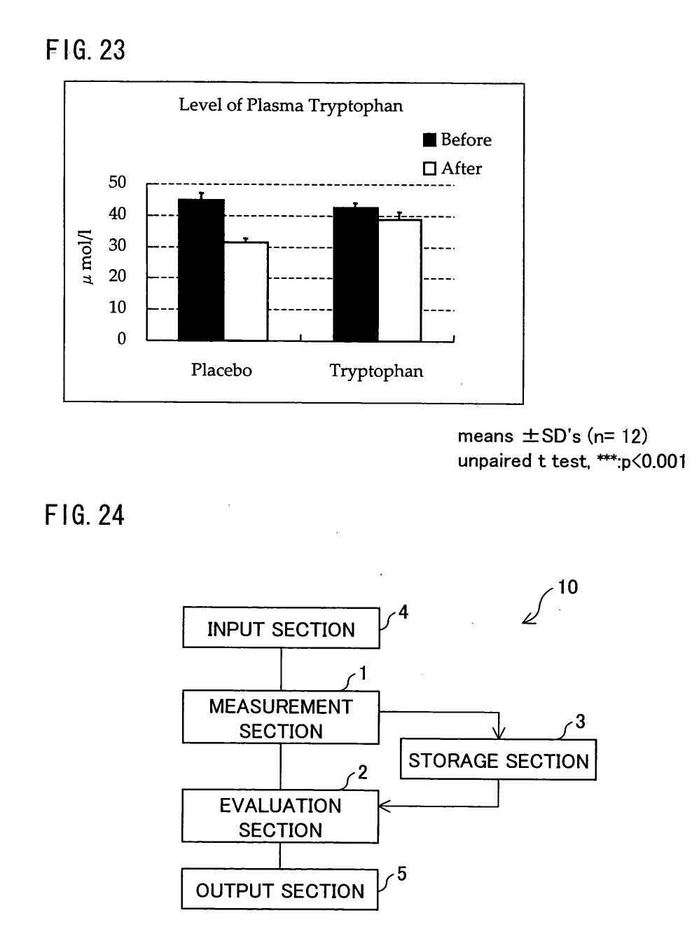

Blood sample was taken from the subjects according to the experimental schedule shown in Table 4 so as to measure concentrations of amino acids in their plasma. FIG. 23 shows a result of measuring concentrations of tryptophan, which is one type of amino acid.

[5-5] Results

In the placebo group, the concentration of tryptophan in plasma before fatigue loading was 45 (μmol/l), and the concentration of tryptophan in plasma after fatigue loading was 31 (μmol/l), so that there was a significant decrease. However, in the tryptophan group, the concentration of tryptophan in plasma before fatigue loading was 43 (μmol/l), and the concentration of tryptophan in plasma after fatigue loading was 39 (μmol/l), so that there was no significant decrease and the concentration of tryptophan in plasma was maintained even after fatigue loading. Because long hours of driving work must have raised the degree of fatigue of the subjects, it became clear that it is possible to evaluate a decrease in concentration of tryptophan in a subject's plasma as being indicative that the subject has a high degree of fatigue.

### INDUSTRIAL APPLICABILITY

As described above, the fatigue evaluation method, the fatigue evaluation kit, and the application thereof according to the present invention bring about an effect of quantitatively evaluating the degree of fatigue of a subject by simply taking plasma from the subject. Furthermore, both of the method and the kit are easy to use and do not require long binding hours, so that a subject is released from trouble and pain and a user can easily use the method and the kit. Both of the method and the kit are easy for both the subject and the user to deal with. Therefore, both of the method and the kit can be applied to a method for screening an anti-fatigue substance and to an in vivo evaluation of a food product or other products featuring an anti-fatigue effect, and are thus very useful techniques.

That is, the fatigue evaluation method according to the present invention can be used to shed light on mechanisms of stress and fatigue, so that a method for releasing stress can be developed and the degree of fatigue can be evaluated. Further, using the present invention makes it possible to quantify (evaluate) effects of health food products, specified health food products, nutrition-supplement drinks, and other products each of which is available in the market and each of which features an anti-fatigue effect. Therefore, the present invention can be applied in a wide variety of fields such as the medical industry, the pharmaceutical industry, the health food industry, and the health appliances industry.

1. A fatigue evaluation apparatus, comprising:
measuring means for measuring a concentration of amino acid in a body fluid; and
evaluating means for evaluating a degree of fatigue by using as an index a measurement result obtained by the measuring means.

2. The fatigue evaluation apparatus according to claim 1, wherein when the measurement result shows that the concentration of the amino acid is lower than a predetermined value, the evaluating means determines that the degree of fatigue is high.

3. The fatigue evaluation apparatus according to claim 1, wherein when the measurement result shows that the concentration of the amino acid is lower than the predetermined value, the evaluating means determines that there is an overwork state due to accumulation of physiological acute fatigue developed in daily life.

4. The fatigue evaluation apparatus according to claim 1, wherein the body fluid is at least one type of body fluid selected from a group consisting of: blood, saliva, cerebrospinal fluid, and urine, all of which have been separated from an individual organism.

5. The fatigue evaluation apparatus according to claim 1, wherein the amino acid is at least one type of amino acid selected from a group consisting of: total amino acids, branched-chain amino acids, aromatic amino acids, cysteine, methionine, lysine, arginine, and histidine.

6. The fatigue evaluation apparatus according to claim 1, wherein a target for evaluation of the degree of fatigue is physiological acute fatigue developed substantially from mental fatigue.

7. The fatigue evaluation apparatus according to claim 1, wherein:
the measuring means measures respective concentrations of the amino acid in the body fluid before and after a subject is subjected to fatigue loading, and
the evaluating means evaluates the degree of fatigue by using as an index a change in concentration of the amino acid in the body fluid between before and after the fatigue loading, based on the measurement result obtained by the measuring means.

8. A fatigue evaluation method for evaluating a degree of fatigue in an organism, comprising:
using an index indicative of a concentration of amino acid in a body fluid from the organism.

9. The fatigue evaluation method according to claim 8, wherein when the index is indicative of the concentration of the amino acid being low, it is determined that the degree of fatigue is high.

10. The fatigue evaluation method according to claim 8, wherein when the index is indicative of the concentration of the amino acid being low, it is determined that there is an overwork state due to accumulation of physiological acute fatigue developed.

11. The fatigue evaluation method according to claim 8, wherein the body fluid is at least one type of body fluid selected from: blood, saliva, cerebrospinal fluid, and urine.

12. The fatigue evaluation method according to claim 8, wherein the amino acid is at least one type of amino acid selected from: total amino acids, branched-chain amino acids, aromatic amino acids, cysteine, methionine, lysine, arginine, and histidine.

13. The fatigue evaluation method according to claim 8, wherein said index is for identifying physiological acute fatigue developed substantially from mental fatigue.

14. The fatigue evaluation method according to claim 8, wherein the degree of fatigue is evaluated by using as an index a change in concentration of the amino acid in the body fluid of the organism between before and after fatigue loading.

15. The fatigue evaluation method of claim 8, further including providing a fatigue evaluation kit for carrying out the fatigue evaluation method.

16. A method for measuring an anti-fatigue effect of an anti-fatigue substance on a subject, comprising:
determining a first measurement indicative of a first concentration of amino acid in the body fluid of the subject:
determining a second measurement indicative of a second concentration of amino acid in the body fluid of the subject after the subject is subjected to the fatigue loading;
administering the anti-fatigue substance to a subject in a fatigue state; and
measuring the anti-fatigue effect of the anti-fatigue substance by using a result indicative of a discrepancy between the first and second measurements.

17. A method for evaluating an anti-fatigue effect of an anti-fatigue substance, comprising:
administering the anti-fatigue substance to a subject in a fatigue state;
measuring a concentration of amino acid in a body fluid of the subject; and
evaluating the anti-fatigue effect of the anti-fatigue substance by using a concentration measurement resulting from said step of measuring as an indicator of a degree to which the subject has recovered from fatigue.

18. An anti-fatigue effect evaluation system, comprising:
a first fatigue evaluation apparatus for evaluating a degree of fatigue of a subject to whom an anti-fatigue substance has been administered; and
a second fatigue evaluation apparatus for evaluating an anti-fatigue effect of the anti-fatigue substance by using as an index a degree to which the subject has recovered from fatigue, based on an evaluation result obtained by the first fatigue evaluation apparatus;
wherein at least one of the first and second fatigue evaluation apparatuses measure a concentration of at least one amino acid in fluid from the subject;
wherein the fluid includes one of: blood, saliva, cerebrospinal fluid, and urine; and
wherein the at least one amino acid is selected from a group consisting of: total amino acids, branched-chain amino acids, aromatic amino acids, cysteine, methionine, lysine, arginine, and histidine.

19. A method for screening a candidate substance for an anti-fatigue substance, the method comprising the processes of:
administering a test substance to a model animal in a fatigue state;
determining whether or not the model animal has recovered from fatigue, by measuring a concentration of amino acid in a body fluid of the model animal; and determining that the test substance includes a candidate substance for an anti-fatigue substance, by using as an index the model animal's recovery from fatigue.
